(19) United States
(12) Patent Application Publication
(10) Pub. No.: US 2013/0096501 A1
(43) Pub. Date: Apr. 18, 2013

(54) System and Method for Patient Access Port Reporting

(71) Applicants: Lawrence Arne, Palo Alto, CA (US); Todd Thompson, San Jose, CA (US); Mark Zdeblick, Portola Valley, CA (US); Angela Strand, Portland, OR (US); George Savage, Portola Valley, CA (US); Yuriy Mestman, Mountain View, CA (US); William H. McAllister, Saratoga, CA (US)

(72) Inventors: Lawrence Arne, Palo Alto, CA (US); Todd Thompson, San Jose, CA (US); Mark Zdeblick, Portola Valley, CA (US); Angela Strand, Portland, OR (US); George Savage, Portola Valley, CA (US); Yuriy Mestman, Mountain View, CA (US); William H. McAllister, Saratoga, CA (US)

(73) Assignee: PROTEUS DIGITAL HEALTH, INC., Redwood City, CA (US)

(21) Appl. No.: 13/805,987

(22) PCT Filed: Jun. 20, 2011
(86) PCT No.: PCT/US2011/041115

§ 371 Date: Dec. 20, 2012

Related U.S. Application Data
(60) Provisional application No. 61/357,506, filed on Jun. 22, 2010.
(60) Provisional application No. 61/377,072, filed on Aug. 25, 2010.

(51) Int. Cl. A61M 5/50

(52) U.S. Cl. CPC A61M 5/5086
USPC 604/111

(57) ABSTRACT

A method is disclosed for detecting, by a sensing and data acquisition circuit module, at least one parameter associated with an access port. The method includes wiping at least a portion of an access port, allowing the access port to dry for a predetermined amount of time, accessing the access port with an access device, such as a syringe, and detecting at least one parameter associated with the access port by the sensor and acquisition module. An access port is disclosed that includes a sensor and a data acquisition module for sensing, recording, and providing an alert associated with the detection of the at least one parameter associated with the access port.

### CROSS REFERENCE TO RELATED APPLICATIONS

This application claims the benefit of U.S. Provisional Patent Application Ser. No. 61/357,506, filed Jun. 22, 2010 and entitled “System and Method for Detection of Delivery of an Inhalable Dose,” and U.S. Provisional Patent Application Ser. No. 61/377,072, filed Aug. 25, 2010 and entitled “System and Method for Patient Access Port Reporting,” each of which is hereby incorporated by reference in its entirety.

### CROSS REFERENCE TO RELATED APPLICATIONS

In various aspects, the present invention is related to electronic systems and methods for patient access port reporting.

### INTRODUCTION

Commonly used inhalable dose delivery systems include a chamber for storing the dose. As needed, a patient will use the system to take and inhale a dose of the medication. However, there is no accurate way of determining if the patient took the inhalable dose or when the patient took the dose. For example, there are delivery systems that track the number of times a dose was delivered. However, the delivery systems lack the ability to determine when the doses were delivered. For example, a patient may discharge the delivery system several times in a short period of time to give the appearance that several doses were taken over a period of time. There are some delivery systems that time-stamp the delivery, but lack the ability to determine if the patient actually received the dose as required. For example, the patient may discharge the delivery system over a long period of time without actually taking or inhaling the dose to give the appearance that the doses were taken regularly over a longer period of time. Thus, these delivery systems are not capable of determining if the patient actually received the dose. Furthermore, there is no accurate way of determining if the dose that was delivered was actually delivered to the patient as intended. For example, the delivery system may have been discharged to someone other than the patient. Additionally, in some instances, the delivery systems may have been accidentally discharged.

A user or patient in a hospital or in home care situations often have an access device associated with the user or placed within the user for periods of time ranging from hours to many days, weeks or even months. The access device defines an access port or opening for many purposes that include both the introduction and removal of fluid from the body. The access device can be used to inject or infuse medication and basic fluids as well as removing fluids such as blood and waste. Typical examples of an access device 100 includes: Catheters (venous, urinary, etc,), Intravenous Access ports, ostomy ports, etc.

With all of such access devices there are high incidences of infections that result from general use and cleaning. The proper care of an access device involves the proper cleaning and disinfecting of the port before it is used, especially for an access device that remains in place for more than a single use. In its most basic form, the cleaning may involve the act of wiping the access port with a disinfectant like alcohol before inserting a syringe or other device into the access port for introducing or removing fluids from the body. There are generally accepted good practices for this activity which involve proper technique and time spent with each activity. In particular, in order to help prevent Hospital Acquired Infections, a standard has been mandated by the Joint Commission on the Accreditation of Healthcare Organizations (JCAHO) for the process of disinfecting an access port. The general process include three major steps: (1) wipe the access port with an alcohol swab for minimum of 30 seconds; (2) allow the access port to dry for 30 seconds; (3) access the port with a syringe within approximately 60 to 120 seconds of the completed drying time.

Therefore what is needed is a system and method for detecting and reporting the cleaning of the access device in accordance with accepted best practices. In a general sense, there is a need for detecting the presence of a disinfectant at the access port site and/or detecting a physical activity or action associated with cleaning or disinfecting the access port. In either case, there is also a need to record the detection to confirm that the access device was cleaned in accordance with accepted best practices.

### SUMMARY

In accordance with the systems and teaching of various aspects of the present invention, a system and method are provided. The method includes detecting, by a sensing and data acquisition circuit module, at least one parameter associated with an access port. The system includes a sensing and data acquisition circuit module configured to detect at least one parameter associated with an access port. The sensing and data acquisition circuit module further includes at least one sensor to sense the at least one parameter associated with the access port and generate a signal corresponding to the at least one parameter associated with the access port and a processor coupled to the sensor to process the signal generated by the sensor.

### BRIEF DESCRIPTION OF THE FIGURES

FIG. 1A is an illustrative example of a user wearing a detector and making contact with a device, which is shown in block diagram form, which delivers an inhalable dose in accordance with the teachings of various aspects of the present invention.

FIG. 1B shows one possible physical shape for the device of FIG. 1, in accordance with the teachings of various aspects of the present invention.

FIG. 1C shows one possible physical shape for the device of FIG. 1, in accordance with the teachings of various aspects of the present invention.

FIG. 1D shows one possible physical shape for the device of FIG. 1, in accordance with the teachings of various aspects of the present invention.

FIG. 1E is an illustrative example of a user wearing a detector and making contact with a device, which is shown in block diagram form, which delivers an inhalable dose in accordance with another aspect of the present invention.

FIG. 1F is a side view illustration of a portion of one aspect of the device of FIG. 1D.

FIG. 2A is an alternative aspect of the device of FIG. 1 shown in block diagram form and in accordance with the teachings of various aspects of the present invention.

FIG. 2B is an alternative aspect of the device of FIG. 1 shown in block diagram form and in accordance with the teachings of various aspects of the present invention.

FIG. 2C is a top view illustration of an acoustic device that in accordance with one aspect of the present invention.

FIG. 2D is an illustration of an acoustic device in accordance with one aspect of the present invention.

FIG. 2E is an illustration of an acoustic device in accordance with one aspect of the present invention.

FIG. 3A is an illustration of the opening of the device of FIG. 1 in accordance with another aspect of the present invention, wherein a diaphragm is positioned near the opening of the device through which the inhalable dose is dispensed.

FIG. 3B is an illustration of one aspect of the diaphragm of FIG. 3, wherein the diaphragm portions are parted as the dose is inhaled by the user such that a beam of light is interrupted and the interruption is detected.

FIG. 3C is an illustration of one aspect of the diaphragm of FIG. 3 being parted as the dose is inhaled by the user such that a flexing motion and parting of the diaphragm portions cause an interruption in a connection.

FIG. 4 is a block diagram illustration of one aspect of the detector of FIG. 1.

FIG. 5 is a block diagram illustration of one aspect of a processing unit of FIG. 4.

FIG. 6 shows example of a user wearing a detector and making contact with a device as the user inhales to receive an inhalable dose in accordance with the teachings of various aspects of the present invention.

FIG. 7 shows one aspect of an access device with the ability to record the timing of certain events associated with maintaining the access device.

FIG. 8 illustrates one aspect of a sensor and data acquisition module coupled to an access port for detecting the action of wiping the access port.

FIG. 9 illustrates one aspect of an access port with a conductive trace.

FIG. 10 illustrates one aspect of a sensor arrangement for detecting the presence of a cleaning substance at an access port site.

FIG. 11 illustrates one aspect of a sensor arrangement for detecting a physical action associated with cleaning an access port.

### DETAILED DESCRIPTION

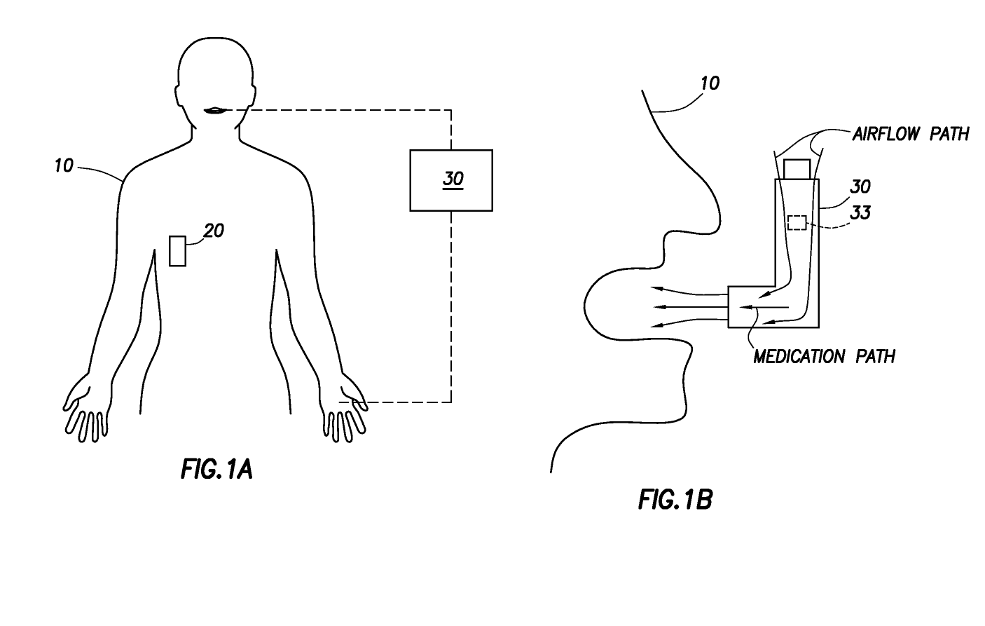

Referring to FIG. 1A, a user 10 is shown wearing a detector 20 and making physical contact with a device 30; the detector 20 and device 30 are described in greater detail below. The detector 20 is shown secured at one location on the user's body. However, the scope of various aspects of the present invention is not limited by the positioning of the detector 20 on the user's body. The detector 20 may be secured to any location on the user's body. Additionally, in the present example, the detector 20 is shown as a detector that is external to the user's body. In accordance with another aspect of the present invention, the detector 20 may be positioned or implanted within the user's body. In yet another aspect of the present invention, the detector 20 may be partially implanted within the user's body. The externally secured detector 20 of interest includes those that are sized to be stably associated with a living subject in a manner that does not substantially impact movement of the living subject. As such, the detector 20 may have dimensions that, when secured to the subject, such as the user, will not cause the subject to experience any difference in mobility or movement. In accordance with some aspects of the present invention, the detector 20 is dimensioned such that its size does not hinder the ability of the subject to physically move. For example, the detector 20 has a small size and may occupy a volume of space of 25 cm³ or less, such as 12 cm³ or less, including 5 cm³ or less. In some instances, the receiver has a chip size limit ranging from 10 mm² to 2 cm² with negligible thickness.

In one aspect, the user 10 holds the device 30 in the user's hand and places the device 30 to the user's mouth, thereby making contact with the device 30 in at least two locations, as shown. The device 30 is shown in block diagram form for clarity of describing the functionality thereof as well as the components therein. However, the device 30 can be made is a variety of shapes according to the various aspects of the present invention.

Referring now to FIGS. 1B, 1C, and 1D, in accordance with various aspects of the present invention, the device 30 can be in a variety of shapes. For example, FIG. 1B shows the device 30 as an upright inhaler with a chamber that is capable of receiving a pressurized can that contains multiple, single doses, each dose delivered individually over a period of time to the user 10. FIG. 1C shows the device 30 as a single dose inhaler with a chamber that is capable of holding one dose. After the single dose is delivered to the user 10, the chamber is reloaded. FIG. 1D shows the device 30 as an inhaler capable of providing continuous delivery of a dose or delivery of a dose at pre-determined intervals using a motorized unit 31 that is plugged into a power source. Additional examples and shapes for the delivery of an inhalable dose are considered and within the scope of various aspects of the present invention. Thus, according to various aspects of the present invention, the device 30 can be in various shapes and dose delivery set-ups, as shown in FIGS. 1B, 1C, and 1D and the scope of various aspects of the present invention is not limited by the actual shape or dose-delivery type or dose-delivery timing of device.

In each of the examples of the device 30, each device includes an acoustic device 33 in accordance with various aspects of the present invention. In FIG. 1B the acoustic device 33 is shown in the path of the air flow but not in the path of the medication. In FIG. 1B the acoustic device 33 is shown in the path of the air flow and the medication. In FIG. 1D the acoustic device is shown away from the path of the air flow and the medication. The scope of various aspects of the present invention is not limited by the location of the acoustic device relative to the path of the air flow and medication flow.

Referring now to FIG. 1E, the user 10 is shown making contact through a contact area 30d to a device 30a is shown having a signal source 30b, a capacitive coupler 30c and contact area 30d. The capacitive coupler 30c is electrically isolated from the contact area 30d. The contact area 30d is an electrode or plate that represents one side of a capacitor relative to ground. The user 10 is also wearing a detector 20a that includes a capacitive coupler 20c. A capacitive conductance path is shown with a broken line in FIG. 1E between the capacitive coupler 30c and 20c. In the figure, the physical contact between the user 10 and the device 30a is shown by the line AA. It will be apparent that the contact between the user 10 and the device 30a is actually a physical contact and it can occur between the mouth or hand of the user 10 and the device 30a. The scope of various aspects of the present invention is not limited by the area on the device 30a that makes contact with the user 10. For example, any part of the user 10 that makes contact with the contact area 30d is within the scope of various aspects of the present invention.

Referring now to FIG. 1E and FIG. 1F, the detector 20a is shown in an exploded view at a skin location 20b of the user 10. The capacitive coupler 20c includes a contact area 20d that is in contact or near contact with the user 10 at skin location 20b and a non-contact side 20e. Through the body of the user 10 and the contact area 30d, the source 30b is physically coupled to the contact side 20d. Line BB is shown to illustrate an electrical connection between the patch in accordance with one aspect of the present invention, through the air and/or ground, the capacitive coupler 30c is coupled to the capacitive coupler 20c of the detector 20a. Capacitive coupling occurs between the device 30a and the detector 20a. Hence, in accordance with another aspect of the present invention, the device 30a includes one contact area 30d that comes into contact with the patient while a second contact area, the capacitive coupling area 30c, is isolated from the patient. The device uses a capacitive return path to the receiver via space or ground.

Thus, by controlling the isolating characteristics of the source 30a, using a control module of the device 30, information is encoded in a carrier wave. For example, using Frequency-shift Keying (FSK) information may be encoded in the carrier wave, including binary FSK.

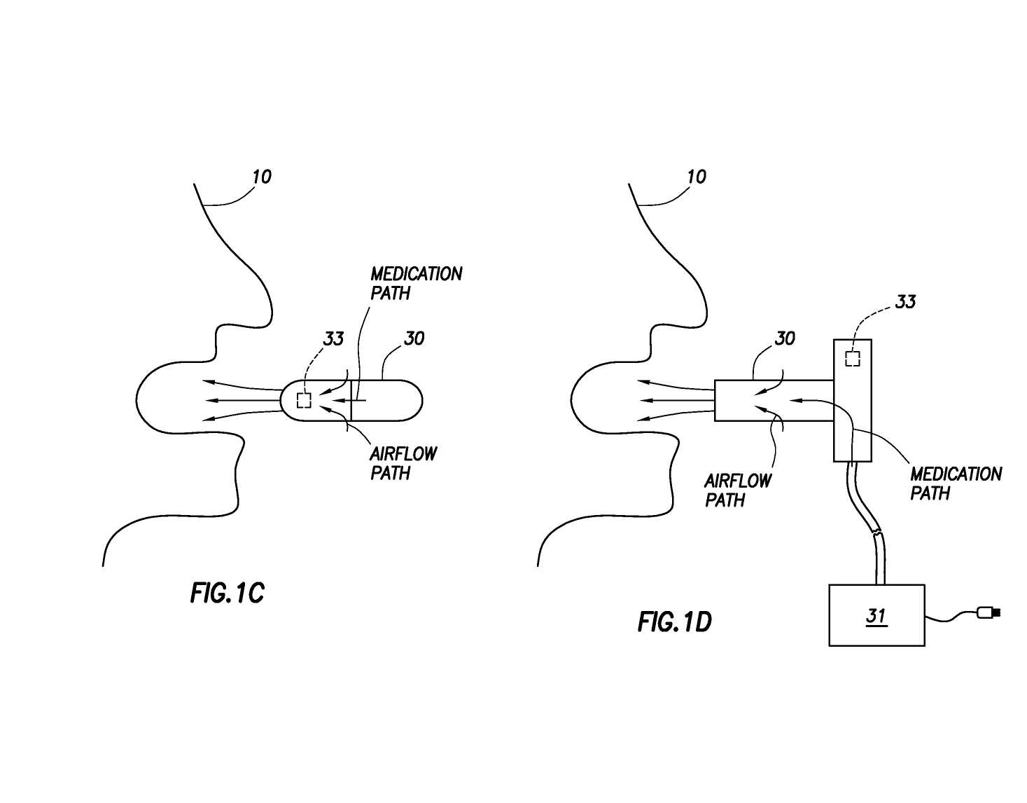

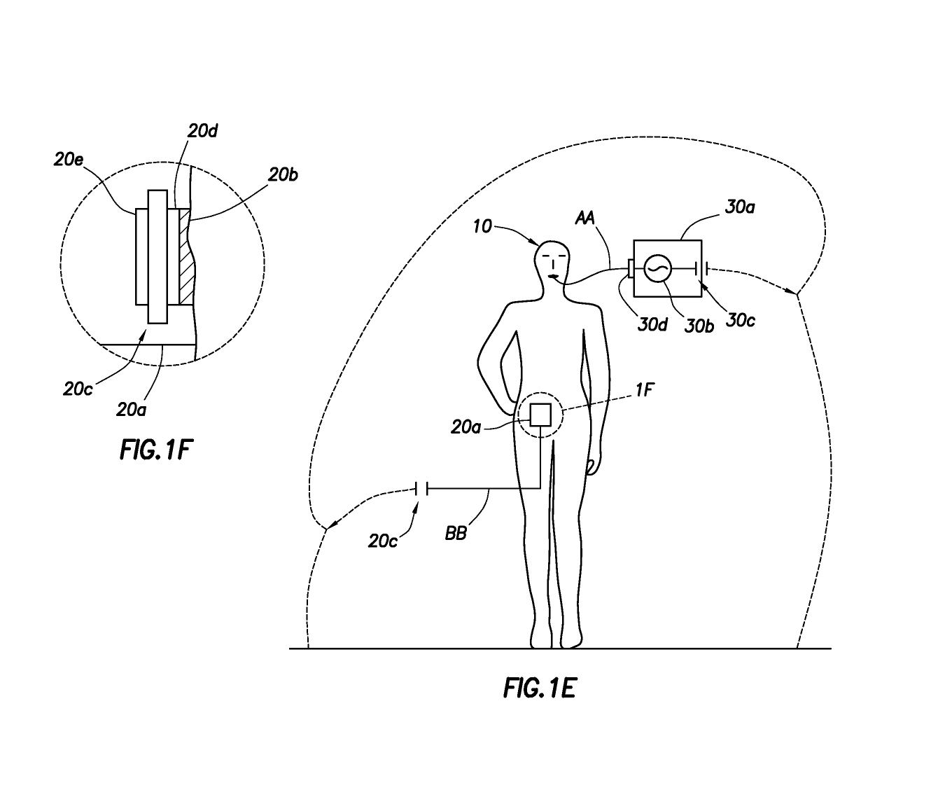

Referring now to FIG. 2A, the device 30 includes a housing 32, the acoustic device 33, a power source 34, a control unit 36, contact areas 38 and 40 and a memory unit 42. The housing 32 defines a chamber 44 for holding the inhalable doses. The chamber 44 may also include a chamber control unit for controlling dispensing of the dose. The chamber 44 also includes an opening, as discussed with respect to FIG. 3 below, through which the inhalable dose is delivered to the patient. In accordance with one aspect of the present invention, the housing 32 includes at least two contact areas/points 38 and 40 positioned at different locations on the housing. The location and position of the contact points or areas are determined by the shape and design of the device 30 and in accordance with the various aspects of the present invention. Multiple locations for the contact areas 38 and 40 are contemplated.

The contact areas 38 and 40 are electrically isolated from each other and at least partially exposed to allow the user 10 to make contact therewith. In accordance with one aspect of the present invention, the contact areas 38 and 40 are positioned such that one contact area makes contact with the user's hand and the other contact area makes contact with the user's mouth. In accordance with alternative aspect of the present invention, additional contact areas may be added to allow for secondary contacts with the hand or mouth as well as to accommodate using a different hand, such as a left hand grip as well or a right hand grip. Furthermore, additional contact areas on the housing can be included to ensure that the device is held properly.

The power source 34 is electrically connected to the acoustic device 33, the control unit 36, and coupled to the contact areas 38 and 40. The acoustic device 33 is also electrically coupled to the control unit 36 and the operation thereof is discussed with respect to FIGS. 2C, 2D, and 2E. One terminal of the power source 34 is electrically connected to the contact area 40. The other terminal of the power source 34 is electrically connected to the control unit 36. As shown, the power source 34 has two outputs; one output is electrically connected to the control unit 36 and the output is connected to the contact area 38. In this example, the control unit 36 is connected in series with the power source 34. However, the scope of various aspects of the present invention is not limited by the relative circuit relationship between the control unit 36 and the power source 34. For example, the control unit 36 may be positioned in parallel with the power source 34, such that the control unit 36 is electrically connected to both of the contact areas 38 and 40 as well as both terminals of the power source 34.

As the device 30 is brought into contact with the user's hand and mouth, the contact areas 38 and 40 come into contact with the user 10 resulting in a complete circuit that includes the user 10. Thus, the circuit that defines the current path includes the user 10, the contact area 40, the power source 34, the control unit 36, and the contact area 38. Once the circuit path is completed the power source 34 provides the voltage potential needed to cause a current flow through the user's body. The presence of the current flow is an indicator that the device 30 is in position for dispensing the inhalable dose because the device 30 is now in the user's hand and has made contact with the user's mouth. The detector 20 identifies the presence of the current flow through the user's body and can record the timing of the event, which is discussed in greater detail below. Additionally, the device 30 can also detect the presence of the current flow and record the time the event occurred. Thus, once the circuit is complete and the current flow is detected, then the control unit 36 can also record the timing of the completed circuit in the memory unit 42.

In accordance with another aspect of the present invention, the control unit 36 provides additional control functionality. According to one aspect, the control unit 36 can control the conductance of the circuit, which is completed through the user's body, to encode information in the current flow. For example, the timing of the completion of the circuit, information about the dose, or additional identifying information can be encoded in the current flow. The control unit 36 alters the conductance of the circuit. The altered conductance results in an alteration of the characteristics of the current flow. It is the altered characteristics that contain the information and, thus, the information is encoded in the current flow, as disclosed in U.S. patent application Ser. No. 12/564,017 entitled COMMUNICATION SYSTEM WITH PARTIAL POWER SOURCE filed on Sep. 21, 2009, and published as 2010-0081894 dated Apr. 1, 2010 the entire specification of which is incorporated herein by reference. The encoded information is then detected by the detector 20 and decoded.

Referring now to FIG. 2B, in accordance with another aspect of the present invention, the control unit 36 can also control communication using additional or alternative communication channels, such as wireless and optical. The control unit 36 is coupled to and in communication with a transceiver 46 that is included in an alternative aspect of the device 30. Although the communication is described in terms of transmissions from the transceiver 46, in accordance with one aspect of the present invention a receiver (not shown) can be included in the device 30 and electrically connected to the control unit 36. Furthermore and in accordance with another aspect of the present invention, the transceiver 46 may be replaced with a transceiver unit that handles both transmission and reception of information. Thus, the control unit 36 allows the device 30 to communicate with the detector 20 using multiple communication channels and methods. In one aspect, the transceiver is configured to communicate with an external device such as a cell phone or a personal computer to indicate that the inhalation event occurred. The wireless communication may be instead of or in addition to generating a signal that indicates the user has inhaled.

In accordance with another aspect of the present invention, the detector 20 communicates identification code or information to the device 30. The identification code or information activates the device 30 to deliver the dose. Additionally, the detector 20 can confirm delivery of the dose and send confirmation information to the device 30 indicating that the dose was delivered.

In accordance with yet another aspect of the present invention, both the detector 20 and the device 30 communicate directly and independently with a third device, such as a cell phone or a computer. The information communicated is related to the delivery of the dose. This third device will then reconcile the data and information to correlate delivery time, dose amount, patient identity, and other related factors, each of which may be received from the detector 20 and/or the device 30.

The device 30 can communicate with various other devices such as a computer with a built-in or peripheral monitor (such as may be found in a bedside monitor or a health information system), a personal digital assistant (PDA), a smart phone, a messaging device, a data center, etc. Additionally, the device 30 may be configured to be interrogated by an external device to provide data to an external location. Any convenient data transmission protocol may be employed, including both conduction through a physical medium (for example, through the user's body using the current flow) and through the air, such as wireless data transmission protocols.

Referring again to FIG. 2B, the control unit 36 is positioned in parallel with the power source 34 because the control unit 36 is connected to each of the contact areas 38 and 40. However, the scope of the present invention is not limited by the relative circuit position of the control unit 36 to the power source 34 within the circuit that is completed through the user's body. For example, the control unit 36 can be positioned in series with the power source 34 such that one output of the power source 34 is connected to the control unit 36 similar to that shown in FIG. 2A. As discussed above, the control unit 36 controls the conductance characteristics of the circuit and hence the characteristics of the current flow. In this manner the control unit 36 can encode information in the current path and allow the current characteristics to carry information to the detector 20.

Referring now to FIG. 2C, the acoustic device 33 includes a magnet 35 and a coil 37. The coil 37 is connected to the control unit 36 of FIG. 2A through connections 33a and 33b. The coil 37 is secured to or mounted on a movable or flexible surface that is positioned proximal to the magnet 35. The control unit 36 measures the current generated as the coil 37 moves within or through the magnetic field associated with the magnet 35. As the relative position of the coil 37 to the magnet 35 is changed, the change in distance results in a change in the characteristic of the magnetic field. Thus, sound waves produced by inhaling or loading the medication for delivery, results in movement of the coil 37 within the magnetic field of the magnet 35. Thus, sound wave can be detected by the control unit 36 through the coil 37.

Referring now to FIG. 2D, the acoustic device 33 is shown in accordance with another aspect of the present invention to include a fixed coil 41 and a movable coil 43. The fixed coil 41 is connected to the power source 34 through connection point 41a and 41b. The power supplied to the fixed coil 41 results in a magnetic field about the fixed coil 41. The movable coil 43 is positioned proximal to the fixed coil 41 and connected to the control unit 36 through connection points 43a and 43b. The control unit 36 detects the presence of the magnetic field associated with the fixed coil 41 through the movable coil 43. As the relative position of the movable coil 43 to the fixed coil 41 changes, the change in distance results in a change in the characteristic of the electromagnetic field associated with the fixed coil 41. As the user 10 dispenses the medication into or inhales through the device 30, the resulting sound waves result in movement of the movable coil 43. Thus, sound waves produced by inhaling or loading the medication for delivery, results in movement of the movable coil 43 within the electromagnetic field of the fixed coil 4. Thus, sound waves can be detected by the control unit 36 through the movable coil 43.

Referring now to FIG. 2E, the acoustic device 33 is shown in accordance with another aspect of the present invention to include a fixed plate 45 and a movable plate 47. The fixed plate 45 is connected to the power source 34 through connection point 45a and 45b. The power supplied to the fixed plate 45 results in a buildup of a charge about the fixed plate 45. The movable plate 47 is positioned proximal to the fixed plate 45 and connected to the control unit 36 through connection points 47a and 47b. The control unit 36 measures the capacitive coupling between the fixed plate 45 and the movable plate 47 through the movable plate 47. As the position of the movable plate 47 relative to the fixed plate 45 changes, the change in distance results in a change in the characteristic of the capacitance associated with the gap AA between the fixed plate 43 and the movable plate 47. As the user 10 dispensed the medication into or inhales through the device 30, the resulting sound waves reach the movable plate 47. Thus, sound waves produced by inhaling or loading the medication for delivery, results in movement of the movable plate 47. Thus, sound waves can be detected by the control unit 36 through the movable plate 47.

One advantage is that the acoustic sensors of various aspects of the present invention may be fabricated at a low cost using manufacturing techniques borrowed from printed circuit board (PCB) fabrication—either by etching or additive/printing. Further advantage may be gained by incorporating the circuits associated with the controller unit, which are in accordance with the various aspects of the present invention, onto the “circuit” board using additive/printing techniques. Furthermore, in accordance with another aspect of the present invention, the power source may be incorporated into the assembly.

In accordance with various aspects of the present invention, the acoustic device 33, as indicated above, may be positioned in various locations within the device, such as out of the air path and the medication path, within the air path and out of the medication path, or within the path of the medication and the air flow. Additionally, in accordance with another aspect of the present invention, the acoustic device 33 may be positioned internal or external to the device 30.

Referring again to FIG. 1A, the detector 20 is shown secured to the subject at one location. The location of the detector 20 is be determined by the medical requirements and the system. The detector 20 employed in accordance with the various aspects of the present invention is configured to be associated with a body location (either inside, partially inside of, or on a surface of a body) and to detect current and electrical signals from one or more devices, such as the device 30 of FIG. 1A. It is also within the scope of various aspects of the present invention to have the detector 20 attached to the clothing of the subject with just electrode leads/wires secured to, or otherwise in contact with, the skin of the subject.

Referring now to FIG. 3A, in accordance with one aspect of the present invention the device 30 is shown with an opening 50 and a diaphragm 52. The diaphragm 52 is shown with six portions 52a, 52b, 52c, 52d, 52e, and 52f. The diaphragm 52 is positioned between the chamber 44 of the device 30 and the opening 50. Thus, as the dose is being dispensed and the user 10 inhales, the portions 52a-f of the diaphragm 52 flex in the direction of the opening 50 and are separated to allow the dose to travel from the chamber 44 through the opening 50 to the user.

Referring now to FIG. 3B, in accordance with one aspect of the present invention, the device 30 includes optical beam sensors 54a and 54b positioned between the diaphragm 52 and the opening 50. As shown, when the diaphragm portions 52a, 52b, and 52c are flexed to allow passage of the dose (as indicated by the direction of the arrows) from the chamber 44 through the opening 50 to the user, the optical beam 56 between the sensors 54a and 54b is interrupted. The interruption or breaking of the beam 56 is caused by the portions 52a-f of the diaphragm. This event is an indication that the dose is being inhaled by the user 10 and the sensors 54a and 54b send a signal to the control unit 36 of the device 30. The control unit 36 can then either encode the information in the current flow or transmit this information to the detector 20 and thereby confirm that the dose was delivered to the user 10.

In accordance with another aspect of the present invention, the chamber 44 may also include an optical gap 58 prior to the diaphragm 52. In the optical gap, at least one optical sensor 60 is positioned to detect an optical emission from a light source 62 also included in the optical gap, such as a Light Emitting Diode (LED). As the dose is released into the optical gap of the chamber 44, the optical sensor 60 detects a drop in the intensity of the light due to the dose being present, which is a powder-like opaque substance. The optical sensor 60 is electrically coupled to the control unit 36. The optical sensor 60 signals the control unit 36 to indicate the presence of the dose once a change in optical intensity is detected.

Referring now to FIG. 3C, in accordance with one aspect of the present invention, the device 30 includes impedance measurement units 58a and 58b positioned between the diaphragm 52 and the opening 50. In the closed position, the diaphragm 52 has a unique impedance when the portions 52a-f are in contact, which is measured by the impedance measurement units 58a and 58b. Once the diaphragm portions 52a-f are flexed and separated, the impedance characteristic of the diaphragm changes. As shown, when the diaphragm portions 52a, 52b, and 52c are flexed to allow passage of the dose (as indicated by the direction of the arrows) from the chamber 44 through the opening 50 to the user, the movement of the diaphragm portions 52a, 52b, and 52c causes a change in impedance of the diaphragm 52. This change in impedance is detected by the impedance measurement units 58a and 58b and is communicated to or signaled to the control unit 36 of the device 30. The control unit 36 can then either encode the information in the current flow or transmit this information to the detector 20 and thereby confirm that the dose was delivered to the user 10.

In accordance with another aspect of the present invention, the device 20 includes a capacitive tactile sensor control unit (not shown). Once the device 20 comes into contact with the user's hand and mouth, then the control unit 36 receives a signal from the sensor control unit and the control module activates a chamber control unit. The chamber control unit initiates dispensing of the dose. According to one aspect to the present invention, the device 20 continues to dispense the inhalable dose to the user 10 such that the device 20 is dispensing a continuous dose. According to another aspect of the present invention, the device 20 dispenses a single dose. Once the sensor control unit detects that the device 20 is no longer in contact with the mouth or hand of the user, then the sensor control unit sends a deactivate or second signal to the control unit 36. The control unit 36 in turn signals the chamber control unit to stop dispensing the dose.

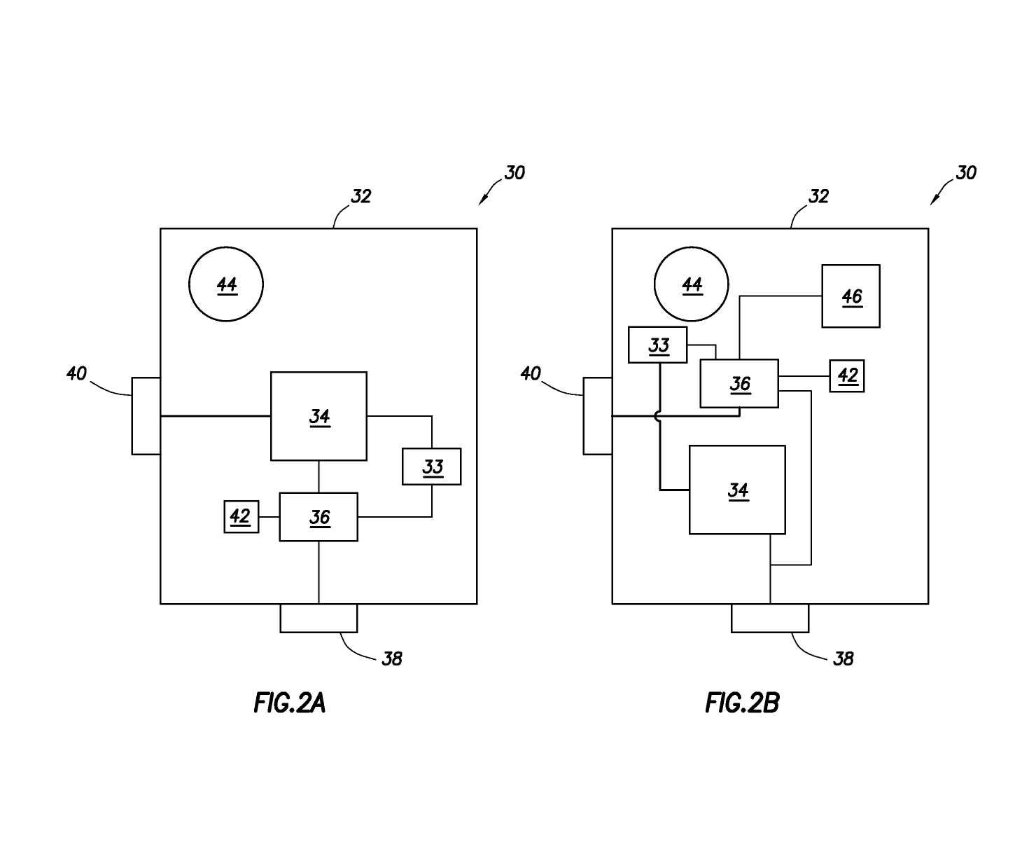

Referring now to FIG. 4, in one aspect, the detector 20 includes a processing unit 70 positioned in a housing 72. The processing unit 70 is electrically coupled to and connected to partially exposed electrodes 74. A coil 76 is wrapped around the housing 72 and electrically coupled to the processing unit 70. The coil 76 is wound around the perimeter and acts as an antenna for signal transmission and reception by the detector 20. In the current example, the detector 20 includes two electrodes. However, in accordance with another aspect of the present invention, the detector 20 may include fewer or greater electrodes and the scope of various aspects of the present invention is not limited by the number electrodes associated with the detector 20. Thus, in one configuration according an aspect of the present invention, the detector 20 includes one or more electrodes (such as two or more electrodes, three or more electrodes, and/or includes multiple pairs of electrodes) for detecting the current signature traveling through the user's body from the device 30. In one configuration of interest, the detector 20 includes two electrodes that are dispersed at a distance “X” from each other, which distance may be one that allows the electrodes to detect a differential voltage potential. This distance may vary, and may range from 0.1 to 5 cm, such as from 0.5 to 2.5 cm. The detector 20 may include a variety of different types of signal receiver elements and processing protocols. Additionally, the detector 20 may be either external to the user's body or implantable.

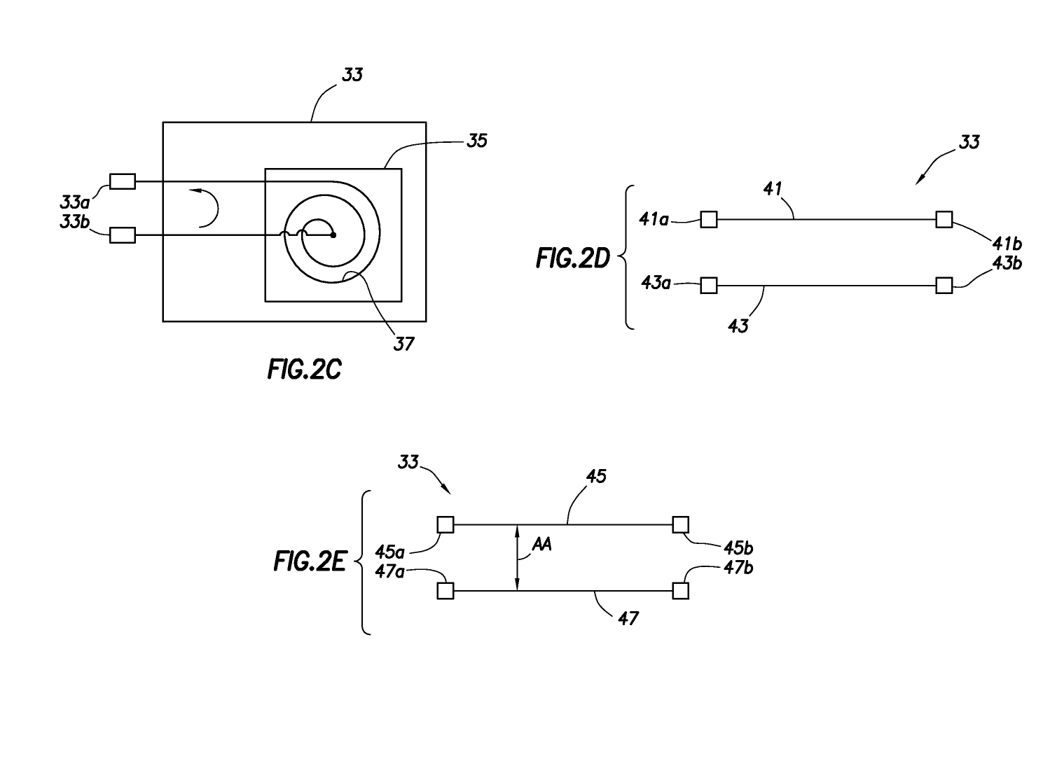

Referring now to FIG. 5, the processing unit 70 includes an amplifier 80 that detects the differential voltage potential across the electrodes 74 of FIG. 4. The detected voltage potential, which represents the current characteristics, is sent to the amplifier 80 through leads 82 that are electrically connected to the electrodes 74 of FIG. 4 via the amplifier 80. The detected current characteristics then go into the demodulator 84. Also shown is a memory unit 85 coupled to the demodulator 84, a clock 86, and a transmitter unit 89. The memory unit 85 is capable of storing information, including data associated with the delivery of the dose to the user 10 as well as changes in the user's physiological condition after the dose is delivered to or inhaled by the user. The clock 86 provides timing information and writes to the memory unit 85 in order to time-stamp the events that are recorded in the memory unit 85. The transmitter unit 89 transfers data from the memory unit 85 to an external data processing unit, not shown. In accordance with another aspect of the present invention, the transmitter unit 89 may be replaced by a transceiver that is capable of receiving as well as transmitting information. The processing unit 70 also includes a power source 87 electrically coupled to a microprocessor 88. The microprocessor 88 is electrically coupled to all the components and coordinates the function between the various functional blocks as well as power management. In accordance with another aspect of the present invention, the components of the processing unit 70, including the microprocessor 88, may be all connected to a bus and, hence, interconnected electrically to each other through the bus and controlled by the microprocessor 88.

According to other aspects of the present invention, the overall system that includes the detector 20 and the device 30 communicate and each record a portion of the event associated with delivery of the dose to the user. For example, as shown in FIG. 5, the processing unit 70 includes a module 90. With the detector 20 positioned on the user's body proximal to the lungs, the act of inhaling can be detected by the module 90, wherein the module 90 is an accelerometer, and the event recorded.

According to another aspect of the present invention, the processing unit 70 of the detector 20 wherein the module 90 includes an acoustic unit. The acoustic device 90 would detect the sound associated with the user 10 taking a deep breath. If the detector 20 has detected the presence of the current flow from the device 30 and records the sound associated with a inhaling, then the acoustic device 90 would record the event of inhalation as an indicated of delivery of the dose to the patient.

According to another aspect of the present invention, the processing unit 70 is coupled to the module 90 that includes an optical detection unit. The optical detection unit of the module 90 would be positioned within the detector 20 such that a portion of the housing would be transparent and allowing for an optical beam to reach the optical detection unit 90.

According to another aspect of the present invention, the processing unit 70 of the detector 20 can include any combination of the accelerometer, the acoustic detection unit, and the optical detection unit.

According to various aspects of the present invention, the system of the invention may include a single detector or multiple detectors. For systems that include a single detector, the detector may include three or more distinct electrodes, and may be configured to be positioned in an abdominal or xyphoid region of the subject. The detector of such systems may be positioned at any convenient location, such as the front of a torso, the back of a torso, etc., as desired. In systems that have multiple detectors, each receiver may have a single electrode and such receivers may be in communication with one another to create an array of detectors.

In accordance with another aspect of the present invention, the acoustic detector and the current flow detection may occur within the device 30 and the existence of both the current flow and the acoustic vibration would indicate that the device is being held in position by the user 10 and the dose has been loaded into the chamber.

Additionally, in accordance with another aspect of the present invention the control unit 36 may enter a sleep state to minimize power consumption. In such a condition, the sound vibrations generated by the dose being loaded into the chamber 44 may be used to generate an activation signal that is send from the acoustic device 33 to the control unit 36. The activation signal may be used to place the control unit 36 in an active state from the sleep state. Once activated, the control unit 36 can record additional acoustic information, especially information associated with the user 10 inhaling through the device 30.

In accordance with another aspect of the present invention, an activation signal may be generated from a mechanical action using a mechanical switch that is triggered as the dose is loaded in to the chamber or through a mechanical motion required by the inhaler to arm the dose delivery.

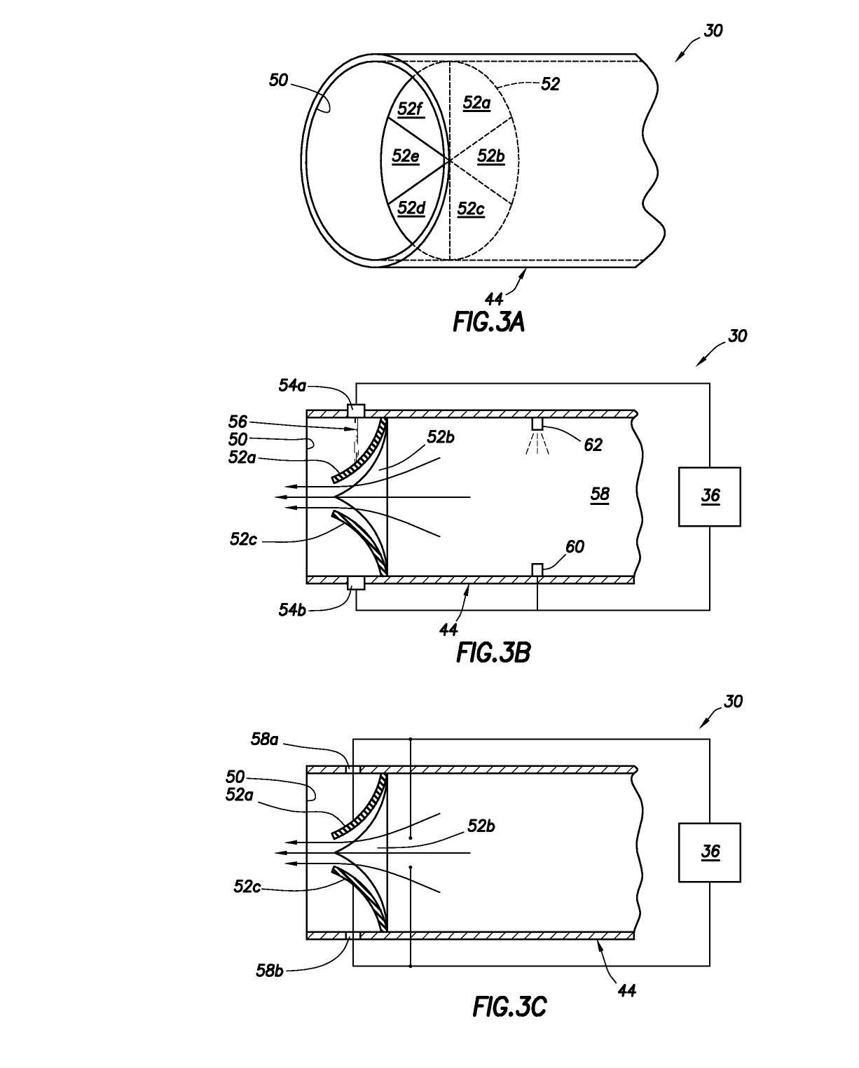

Referring now to FIG. 6, the user 10 places the device 30 to his or her mouth and inhales. In accordance with another aspect of the present invention, the device 30 defines an aperture 310. As the user 10 inhales through the mouth piece 320 of the device 30, the flow of air through the aperture 310 produces a sound, which is similar to the sound produced by a whistle. These sound waves produced by the device 30 travel through the surrounding air as well as into the lungs of the user 10. The sound waves enter the lungs of the user 10 and travel through the user's body. The detector 20 is secured to the user 10 and includes an acoustic detector that detects acoustic waves traveling through the air as well as through the body of the user 10. Thus, as the user 10 inhales, the sound waves produced by aperture 310 of the device 30 can be detected and correlated with the event associated with deliver of the dose to the user 10. The combined detection of the sound wave through the air and tissue of the use is confirmation that the user 10 inhaled through the device 30 and received the dose. Furthermore, in accordance with another aspect of the present invention, the aperture 310 of the device 30 may be adjusted to create a specific or unique frequency that is further used as validation that the user 10 inhaled through the device 30 as expected.

In accordance with another aspect of the present invention, a detector 210 may be used in the form of a watch worn on the wrist of the user 10 as disclosed in PCT Patent Application Serial No. PCT/US11/23017 entitled TWO-WRIST DATA GATHERING SYSTEM filed on Jan. 28, 2011, and in PCT Patent Application Serial No. PCT/US11/23013 entitled DATA GATHERING SYSTEM filed on Jan. 28, 2011.

In accordance with yet another aspect of the present invention, the detector may be implanted and capable of communicating with a detector located external to the body of the user. In this way acoustic waves traveling through the user's body may be detected and correlated with acoustic waves traveling external to the user's body.

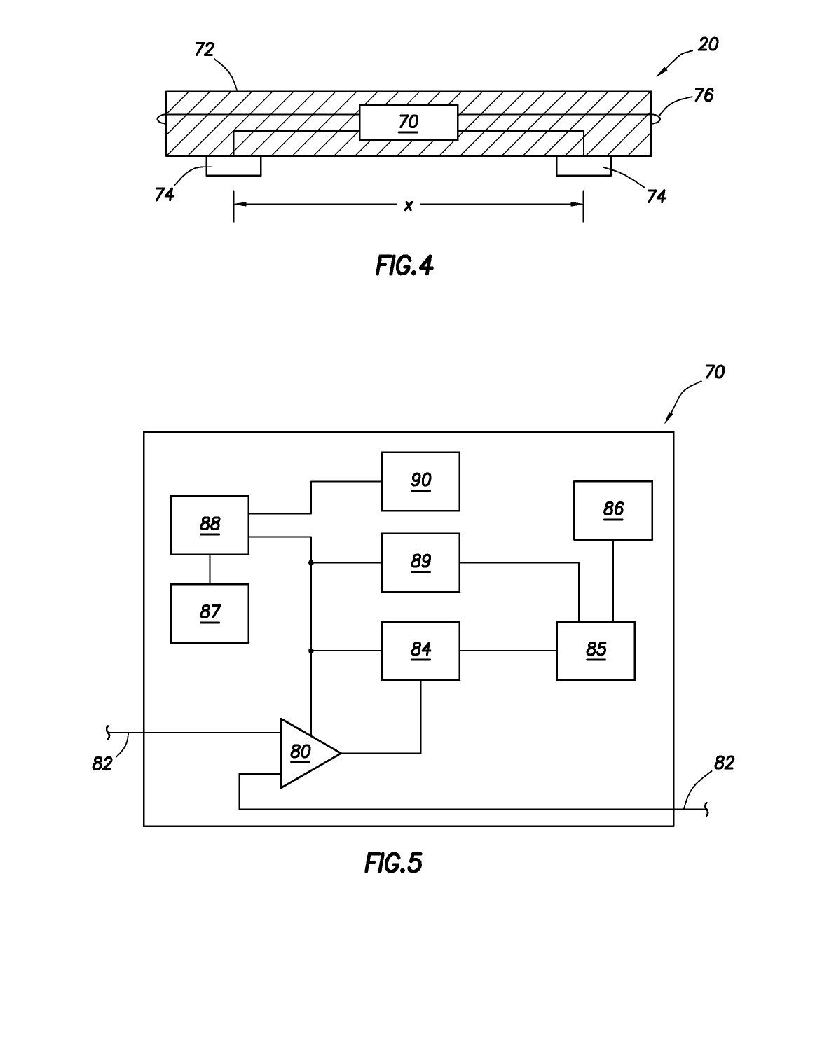

Referring now to FIG. 7, an access device 100 is shown having a needle 102 and a data collection (acquisition) module 104 (circuit) in accordance with another aspect of the present invention. The access device 100 defines an access port or opening 106. In one aspect, the access port 106 may be an access port known under the trade designation of BD Q-Syte by Becton Dickinson and Company of Franklin Lakes, N.J. A user or patient in a hospital or outside of the hospital in home care situations very often has an access device 100, such as the access device 100, placed within the user for periods of time ranging from hours to many days, weeks or even months. The access device 100 is used for many purposes that include both the introduction and removal of fluid from the body. The access device 100 can be used to inject or infuse medication and basic fluids as well as removing fluids such as blood and waste. Typical examples of an access device 100 includes: Catheters (venous, urinary, etc,), IV Access ports, ostomy ports, etc.

With all of these devices there are high incidences of infections that result from general use and cleaning. The proper care of the access device 100 involves the proper cleaning and disinfecting of the port before it is used, especially for the access device 100 that remains in place for more than a single use. In its most basic form, the cleaning involves the act of wiping the access port 106 with a disinfectant like alcohol before inserting a syringe or other device into the access port 106. There are generally accepted good practices for this activity which involve proper technique and time spent with each activity. In particular, in order to help prevent Hospital Acquired Infections, a standard has been mandated by the Joint Commission on the Accreditation of Healthcare Organizations (JCAHO) for the process of disinfecting an access port. The general process include three major steps: (1) wipe the access port with an alcohol swab for minimum of 30 seconds; (2) allow the access port to dry for 30 seconds; (3) access the port with a syringe within approximately 60 to 120 seconds of the completed drying time.

The sensor and data acquisition module 104 is coupled to the needle 102 and the rim of the access port 106. Information regarding the management, cleaning, and use of the access device 100 can be collected (acquired) and stored (recorded) by the sensor and data acquisition module 104. In one aspect, the sensor and data acquisition module 104 comprises a sensor configured to couple to the access port 106. A processing subsystem may be provided, which comprises a processor (e.g., processing unit, microprocessor) to control the operation of the sensor and data acquisition module 104, to read information from the sensor, and to store the readings in a memory unit. A radio subsystem may be provided, which comprises a transceiver to transmit and/or receive information. For example, the information detected by the sensor and recorded in the memory unit may be transmitted or communicated to a detector on the users' body, such as the detector 20 of FIG. 1A. The information can be communicated to the detector using any of the disclosed communication protocols, including controlling the current signature or altering the carrier wave.

In accordance with various aspects of the present invention, the sensor and data acquisition module 104 can be added to the access device 100 as either an add-on device or a redesigned access port that tracks the care of the access port 106. For example, contact with the rim of the access port 106 can be detected and, hence, the time spent cleaning of the access port 106, including the amount of time spent wiping and the drying the cleaned access port 106 is recorded. Furthermore, the time of insertion of an access device 100 such as a syringe or other device into the access port 106 may be recorded as well as the amount of time the access device 100 (e.g., syringe) spends in the access port 106. The collection of data can be used to show that the maintenance of the access port 106 had been done properly and in the proper sequence, and that it was done with each use of the access port 106.

In one general approach, the sensor and data acquisition module 104 may be configured to sense the cleaning or wiping action of the access port 106 using a number of techniques including chemical detection, temperature measurement, impedance measurement, dielectric detection, acoustic measurement, ultrasound measurement, pressure measurement, accelerometer measurement, optical measurement, and magnetic field or magnetic flux detection and/or measurement, among others.

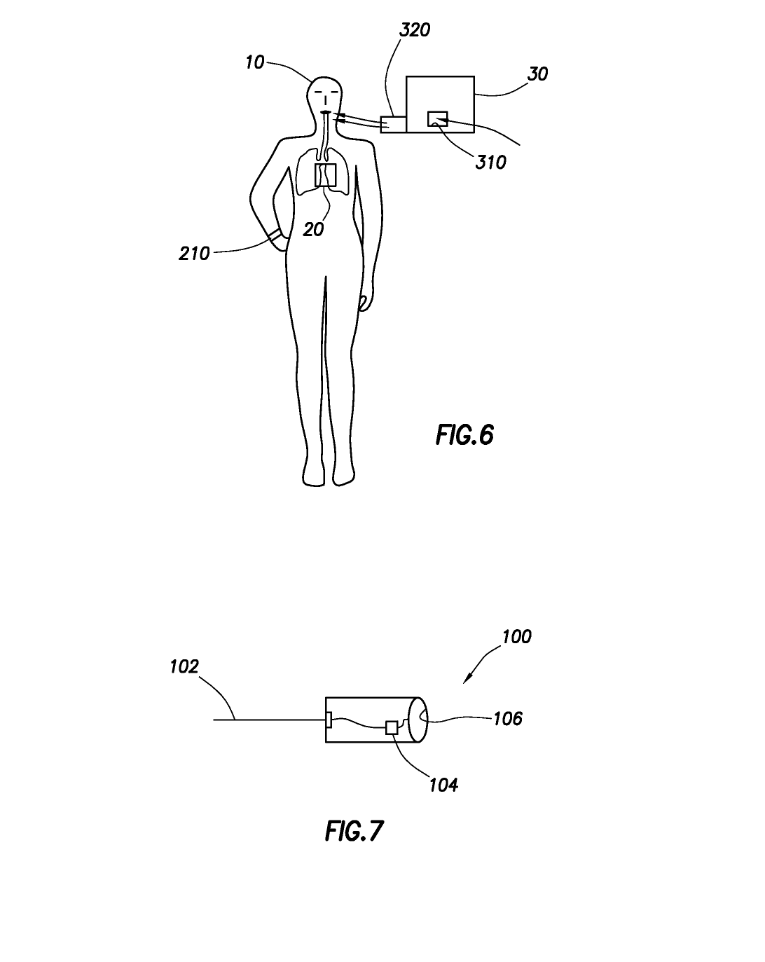

FIG. 8 illustrates one aspect of a sensor and data acquisition module 104 (circuit) coupled to an access port 106 for detecting a cleaning substance at the site of the access port 106 such as a disinfectant and/or an action associated with cleaning the access port 106 such as of wiping the access port 106. In one aspect, the sensor and data acquisition module 104 comprises a sensor 344 configured to couple to the access port 106. A processing subsystem comprises a processor 340 to control the operation of the sensor and data acquisition module 104, to read information from the sensor 344, and to store the readings in a memory unit 342. A radio subsystem comprises a transceiver 346 to transmit and/or receive information. For example, the information detected by the sensor 344 and recorded in the memory unit 342 may be transmitted or communicated to a detector on the user's body, such as the detector 20 of FIG. 1A. An alarm output section 348 (circuit) provides an audible or visual alert. In other aspects, the alarm output section 348 may provide an alert by way of the transceiver 346 or to an external processor/computer.

In one aspect, the sensor and data acquisition module 104 may be employed in a process for detecting a cleaning substance and a physical action associated with cleaning the access port 106 and recording the detection of the cleaning substance and the physical action. Although the cleaning substance is generally applied by wiping the access port 106 with an alcohol swab, other cleaning techniques for cleaning the access port 106 such as spraying a cleaning solution on the access port 106 or dipping the access port 106 in a cleaning solution are contemplated within the scope of the present disclosure. Other techniques including infrared and/or ultraviolet light treatment are also contemplated to be within the scope of the present disclosure. In one aspect, the access port 106 may comprise a sensor and data acquisition module 104. In various aspect, the sensor 344 portion of the sensor and data acquisition module 104 may be configured to sense the cleaning substance or wiping action of the access port 106 using a number of techniques, including without limitation, chemical detection, temperature measurement, impedance measurement, dielectric detection, acoustic measurement, ultrasound measurement, pressure measurement, accelerometer measurement, optical measurement, and magnetic field or magnetic flux detection and/or measurement, among others. In addition, the sensor and data acquisition module 104 is configured to record the detected or measured parameters in the internal memory unit 342. It will be appreciated that the sensor 344 may be selected to suit the particular desired cleaning or wiping method.

As shown in FIG. 8, for example, at 332, the sensor 344 portion of the sensor and data acquisition module 104 senses and records the disinfection of the needle-less access port 106 with an alcohol swab 330 for a predetermined period of time. In accordance with the JCAHO standard, for example, the disinfection period may be approximately 30 seconds. After the predetermined disinfection period expires, the sensor and data acquisition module 104 senses and records air dry for a predetermined period of time sufficient to kill bacteria. Again, in accordance with the JCAHO standard, for example, the air dry period may be approximately 30 seconds. At 334, the sensor and data acquisition module 104 the access port 106 is accessed by an access device 100 such as a syringe within a predetermined period of the completed drying time. In accordance with the JCAHO standard, for example, the access port 106 may be accessed with a syringe within approximately 60 to 120 seconds of the completed drying time. The sensor and data acquisition module 104 senses and records the flush/lock catheter access port 106 regularly with saline/heparin to maintain patency. In addition, the sensor and data acquisition module 104 may sense and record dwell time of the access device 100 at specified time intervals. The sensor and data acquisition module 104 may provide audible or visual alerts based on hospital needs by way of the alarm output section 346.

In various embodiments, the sensor and data acquisition module 104 may be configured for detecting a cleaning substance and wiping action using at least two general approaches. Such approaches may include, for example, detecting the presence of a disinfectant such as alcohol, detecting the effect of the disinfectant, and/or detecting the physical wiping action irrespective of the disinfectant used. It will be appreciated that the sensor and data acquisition module 104 may use any combination of detecting the presence of a disinfectant as well as detecting the physical wiping action. To detect the presence or the effect of a disinfectant such as alcohol on the access port 106, the sensor 344 portion of the sensor and data acquisition module 104 may be configured to detect chemicals, temperature change, impedance change, dielectric change, ultrasound, among others. The sensor 344 may be selected to be suitable for the particular detection technique to be employed.

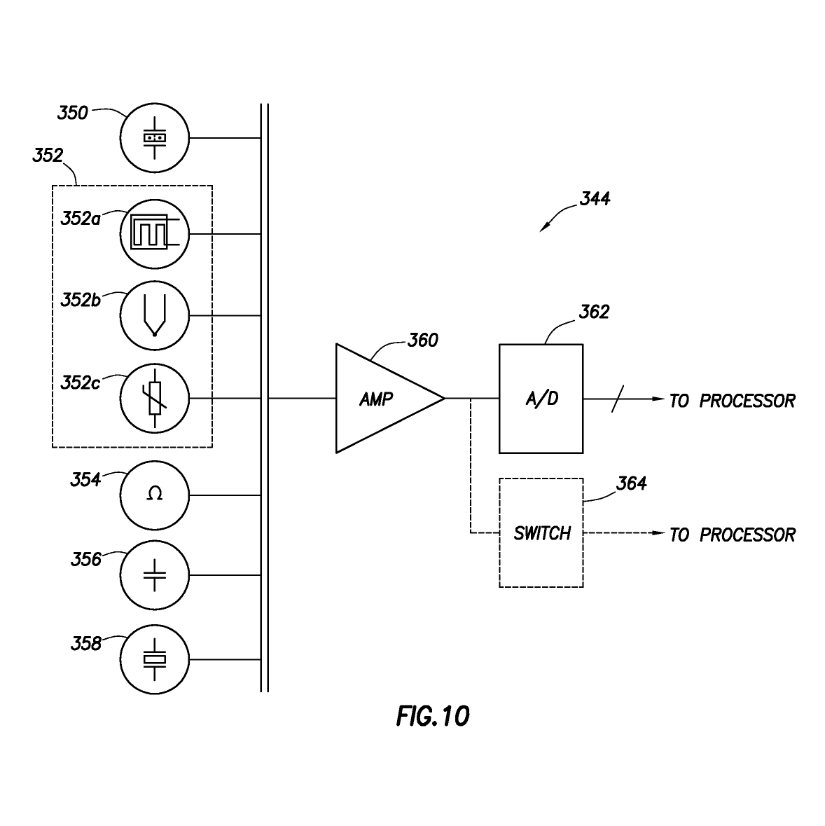

Accordingly, FIG. 10 illustrates one aspect of a sensor 344 arrangement for detecting the presence of a cleaning substance at an access port site. The sensor 344 may comprise one or more sensors. For example, the sensor 344 may be configured to include one or more sensor elements such a chemical sensor 350, a temperature sensor 352 such as a platinum resistance temperature device 352a (RTD), a thermocouple 352b, a thermister 352c. In addition, the sensor 344 may comprise an impedance measurement module 354, a dielectric or capacitive sensor 356, and/or an ultrasound sensor 358, comprising, for example, a piezoelectric transducer. The various sensor elements are located at the access port cleaning site and produce an electrical signal when a cleaning substance is detected at the access port cleaning site. In various aspects, the sensor 344 also may comprise suitable analog and digital electronic circuits to process the electrical signals generated by the sensor elements and provide the information to the processor 340 (FIG. 8). Accordingly, in one aspect, the sensor 344 also comprises an amplifier 360 to receive an electrical signal from a sensor element at an input and provide a conditioned and/or amplified version of the input signal to an analog-to-digital (A/D) converter 362, which then provides a digital version of the signal to the processor 340. The A/D converter 362 may be used when it desirable to measure a quantity of the substance. The processor 340 then records the digitized signal in the memory unit 342 (FIG. 8) along with a time stamp or other indication. In another aspect, the output of the amplifier 360 may be provided to a simple threshold electronic switch 364 or comparator such that when a predetermined threshold is reached, the switch 364 provides an indication to the processor 340, which then records the indication in the memory unit 342.

With reference still to FIGS. 8 and 10, in accordance with various aspects of the present invention, in one aspect, the sensor 344 portion of the sensor and data acquisition module 104 comprises a chemical sensor 350 to detect the presence of a cleaning substance at the access port cleaning site. The cleaning substance may be any suitable disinfectant, such as alcohol. In one example, the chemical detector 350 comprises detecting reagents that are especially sensitive to the isopropyl alcohol (IPA) and quickly produce a distinct layer color change. Such change can be detected either by the human eye or may be converted into an electrical signal, which is provide to the amplifier 360 and then either to the A/D converter 362 for a quantitative measurement or to the electronic switch, which signals when the amplifier 360 produces a signal that exceeds a predetermined threshold. In either case, the processor 340 receives the information and records in the memory 342 along with a time stamp.

With reference still to FIGS. 8 and 10, in another aspect, the sensor 344 portion of the sensor and data acquisition module 104 comprises a temperature sensor 352 (any one of the RTD 352a, thermocouple 352b, thermister 352c) to measure the temperature change on the surface of the access port 106 when a cleaning substance comes in contact therewith. A measured temperature change on the surface of the access port 106 may be correlated with IPA coming in contact with the access port 106 and could be used to detect wiping of the access port 106 with IPA. An experiment was conducted where an alcohol swab 330 held at room temperature was used to wipe the access port 106 while monitoring the temperature of the access port 106 at the cleaning site. The experiment showed that if the alcohol swab 330 was initially at room temperature, little measurable temperature change was shown while rubbing the access port 106 connector. However, upon removal of the alcohol swab 330 or stopping the wiping action altogether, the evaporation of the IPA caused an immediate drop in temperature of approximately 2 degrees F. at the access port 106 cleaning site. It will be appreciated that cooling the alcohol wipes 330 before use would produce a more dramatic temperature signature both during the wiping action and at the cessation of the wiping action, making the temperature change easier to detect by the temperature sensor 352. As previously discussed, the amplifier 360 conditions and amplifies the signal and [provides either to the A/D converter 362 or to the switch 364. In either case, the measured or detected temperature change indicated by the temperature sensor 352 is recorded on the memory unit 342 by the processor 340.

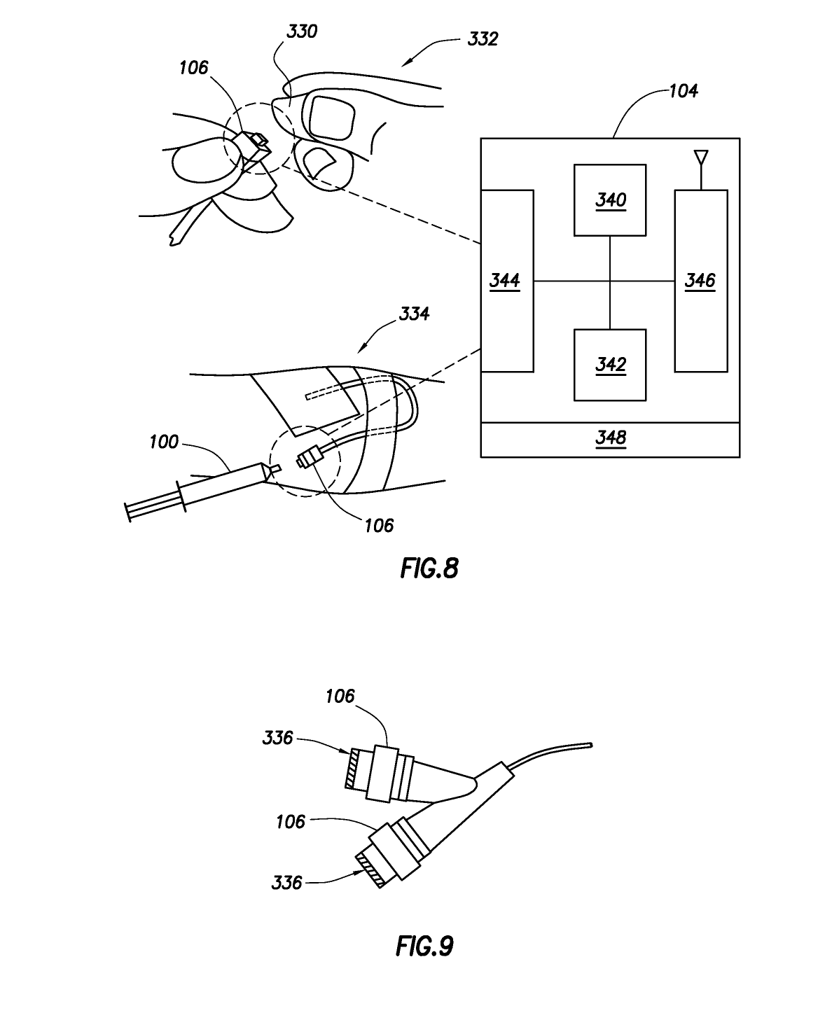

With reference still to FIGS. 8 and 10, in yet another aspect, the sensor 344 portion of the sensor and data acquisition module 104 comprises an impedance measurement module 354 to detect a change in impedance at the access port 106 cleaning site when it comes into contact with a cleaning substance. The use of an impedance change across the surface of the access port 106 as a result of the wiping action with an IPA type alcohol swab 330 is another way of detecting the start and completion of the disinfecting. In one aspect, a conductive ink/epoxy trace was provided surrounding the rim of the access port 106. Gaps may be provided in the conductive trace that can be closed by the conductivity of the alcohol during the wiping action. An access port 106, such as a BD Q-Syte, for example, with a conductive trace 336 is shown in FIG. 9. A dry access port 106 shows no impedance whereas an access port 106 being wiped with an alcohol swab 330 as shown in FIG. 8 shows impedance in the range of approximately 5 to approximately 10 Mega Ohms. This impedance difference is measured by the impedance measurement module 354 and may be used to sense the wiping action with an IPA type swab 330. The same affect may be achieved through the use of a custom swab 330 that contains conductive material embedded therein. In one aspect, the sensor 344 portion of the sensor and data acquisition module 104 may be configured to detect a change in dielectric, for example. In another example, the impedance measurement module 354 may achieve an impedance measurement by first measuring the impedance between electrodes positioned at the access port 106 without the presence of a conducting fluid, such as the alcohol cleaning fluid. As the access port 106 is cleaned with alcohol or similar conducting cleaning fluid, the impedance between the electrodes is altered by the alcohol or other conducting cleaning fluid and the change in impedance can be measured by the impedance measurement module 354 and the timing of the change can be recorded by the processor 340 in the memory unit 342. As the alcohol evaporates and the wiping action is ended, the impedance returns to the previous level.

In another aspect, the sensor 344 portion of the sensor and data acquisition module 104 may comprise a capacitive or dielectric sensor 356 to detect any changes in capacitance at the access port 106 cleaning site when the site comes in contact with a cleaning fluid such as IPA.

In another aspect, the sensor 344 portion of the sensor and data acquisition module 104 may comprise an ultrasound transducer 358 comprising a piezoelectric transducer element to test the presence of an object touching a membrane portion of the access port 106 at the cleaning site other than air. The response to a wipe with an alcohol swab 330 would be different than air and a syringe, for example. Accordingly, such difference may be detected by the sensor and data acquisition module 104.

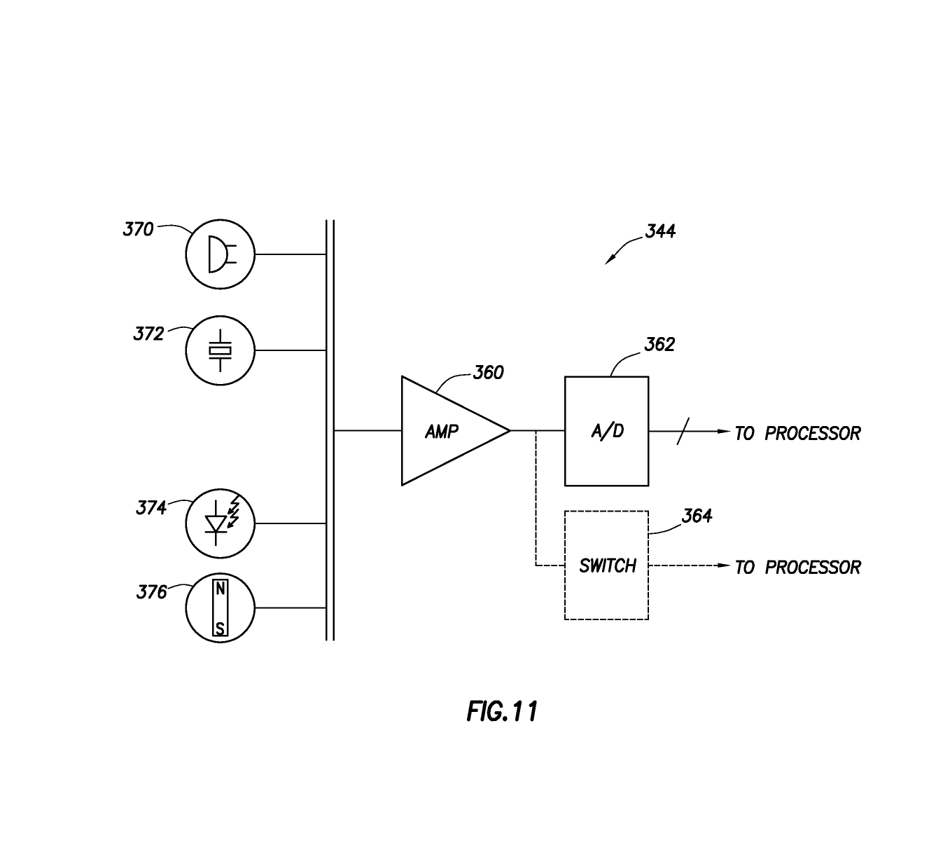

FIG. 11 illustrates one aspect of a sensor arrangement 344 for detecting a physical action associated with cleaning an access port 106. In another approach, the sensor and data acquisition module 104 may be configured to detect a physical wiping action associated with cleaning the access port 106. Accordingly, the sensor 344 portion of the sensor and data acquisition module 104 may be configured to include one or more sensor elements such as an acoustic sensor 370, an accelerometer 372, an optical sensor 374, and/or a magnetic filed/flux sensor 376. As discussed in connection with FIG. 10, the amplifier 360 may be configured to the specific type of sensor signal and then provides the output to the processor 340 either via the A/D converter 362 or the switch 364. The processor 340 records the information in the memory unit 342.

With reference now to FIGS. 8 and 11, in one aspect, the sensor 344 portion of the sensor and data acquisition module 104 may comprise an acoustic sensor 370 to sense or detect the noise or vibration associated with the physical action of wiping the access port 106 with an alcohol swab 330. A custom “noisy” swab may be provided to make this more intense and easier to detect electronically. In accordance with another aspect of the present invention, acoustic detection devices may be used to detect a signature or vibrations associated with the wiping action. Additionally, the sensor and data acquisition module 104 may be configured to detect the pressure associated with the wiping action at the access port 106. The access device 100 makes an electrical connection to the patient to allow the information to be communicated to the detector using alteration of the current signature. In accordance with various aspects of the present invention, the electrical connection between the access device 100 necessary for communication between the data collection module 104 and the detector 20 worn by the user may be made physically or mechanically with any number of methods including: conductive tape, conductive or conductor imbedded tubing.

Still with reference to FIGS. 8 and 11, in other aspects, the sensor 344 portion of the sensor and data acquisition module 104 may comprise an accelerometer 372 to sense any movement associated with the access port 106 while it is being disinfected.

Still with reference to FIGS. 8 and 11, in yet another aspect, the sensor 344 portion of the sensor and data acquisition module 104 may comprise an optical sensor 374 to detect when light is either blocked or reflected by the wiping action thereby signaling the action of wiping the access port 106 and/or to signal the access of the access port 106 by an access device 100 such as a syringe.

Still with reference to FIGS. 8 and 11, in yet another aspect, the sensor 344 portion of the sensor and data acquisition module 104 may comprise a magnetic field/flux sensor 376 to detect a magnetic field or magnetic flux generated by a custom wipe 330 containing magnetic material that could be sensed by sensor and data acquisition module 104 electronics provided in the access port 106.

Accordingly, in conjunction with the sensor and data acquisition module 104, in one aspect, a method is provided for detecting the cleaning and/or wiping action of an access port 106 as shown and described in connection with FIGS. 7-11. In one aspect, a method provides wiping at least a portion of an access port 106, allowing the access port 106 to dry for a predetermined amount of time, accessing the access port 106 with an access device 100, such as a syringe, and detecting at least one parameter associated with the access port 106 by the sensor and acquisition module 104. In another aspect, the method further provides recording the at least one detected parameter associated with the access port 106. In another aspect, the method further provides reporting by the sensor and acquisition module 104 the at least one detected parameter associated with the access port 106. In another aspect, the method further provides providing by the sensor and acquisition module 104 an alert based on the at least one detected parameter.

In one aspect, detecting at least one parameter associated with the access port 106 provides detecting a presence of a wiping solution by the sensor and acquisition module 104. In one aspect, detecting presence of a wiping solution includes detecting via at least one of a chemical detection, a temperature changes, an impedance change, a dielectric, and ultrasound. In another aspect, the method provides detecting at least one parameter associated with an access port by detecting an action associated with wiping of at least a portion of an access port 106, such as a conductive trace 336 as in FIG. 9 formed around the outer surface of the access port 106. In one aspect, detecting an action associated with the wiping of at least a portion of the access port 106 includes detecting via at least one of an acoustic sensor, an accelerometer, an optical sensor, and a magnetic sensor.

In one aspect, the sensor and data acquisition module 104 may comprise hardware and/or software. The memory unit 342 may be divided into data storage memory and software memory. Accordingly, the software may reside in a portion of the memory unit 342. The software in software memory may include an ordered listing of executable instructions for implementing logical functions (i.e., “logic” that may be implemented either in digital form such as digital circuitry or source code or in analog form such as analog circuitry or an analog source such an analog electrical, sound or video signal) executed by the processor 340. Such software may selectively be embodied in any computer-readable (or signal-bearing) medium for use by or in connection with an instruction execution system, apparatus, or device, such as a computer-based system, processor-containing system, or other system that may selectively fetch the instructions from the instruction execution system, apparatus, or device and execute the instructions. In the context of this document, a “computer-readable medium” and/or “signal-bearing medium” is any means that may contain, store, communicate, propagate, or transport the program for use by or in connection with the instruction execution system, apparatus, or device. The computer readable medium may selectively be, for example but not limited to, an electronic, magnetic, optical, electromagnetic, infrared, or semiconductor system, apparatus, device. More specific examples “a non-exhaustive list” of the computer-readable medium would include the following: an electrical connection “electronic” having one or more wires, a portable computer diskette (magnetic), a RAM (electronic), a read-only memory “ROM” (electronic), an erasable programmable read-only memory (EPROM or Flash memory) (electronic).

In accordance with another aspect of the present invention, the communication connection between the detector 20 worn by the user and the data collection module 104 of the access device 100 may be through capacitive conductance using the tubing wall or using the fluid in the tubing as the conductor.

In accordance with other aspects of the present invention, sensors on the access device 100 may be used to verify proper handling or holding of the access device 100 and to activate the data collection module 104 to document the cleaning and use of the access device 100. For example, the device could be designed to verify that the nurse was using gloves when handling and accessing the port.

In accordance with another aspect of the present invention, the data collection module 104 may also be used, through its contact with the user, to measure local skin/body temperature and send a signal to the detector, which would warn of infection.

Aspects of implantable versions of the detector may have a hermetically sealed and biologically compatible cavity defined to act as the enclosure, e.g., Chipskin one or more sensing electrodes, a power source, which could either be a primary cell or rechargeable battery, or one that is powered by broadcasting inductively to a coil. For the external signal receivers, aspects include structures that have electrodes in contact with the user's skin. The communication may be wireless or performed over one or more conductive media, e.g., wires, optical fibers, etc. Where desired, the same electrodes may be used for receiving and transmitting signals.

In certain aspects, the components or functional blocks of the detector and the device are present on integrated circuits, where the integrated circuits include a number of distinct functional blocks, i.e., modules and at least some of, e.g., two or more, up to an including all of, the functional blocks may be present in a single integrated circuit. By single integrated circuit is meant a single circuit structure that includes all of the different functional blocks. As such, the integrated circuit is a monolithic integrated circuit that is a miniaturized electronic circuit (which may include semiconductor devices, as well as passive components) that has been manufactured in the surface of a thin substrate of semiconductor material. The integrated circuits of certain aspects of the present invention may be hybrid integrated circuits, which are miniaturized electronic circuits constructed of individual semiconductor devices, as well as passive components, bonded to a substrate or circuit board.

The detectors of interest include, but are not limited to, those receivers disclosed in: PCT application serial no. PCT/US2006/016370 published as WO 2006/116718; PCT application serial No. PCT/2007/24225 published as WO 2008/063626; PCT application serial no. PCT/US2008/52845 published as WO/2008/095183; PCT application serial no. PCT/US2009/68110 published as WO/2008/075115; the disclosures of which applications are herein incorporated by reference.

In accordance with other aspects of the present invention, the system may include two or more (such as three or more, including four or more) detectors. In such systems, the two or more detectors may be adaptively arranged at any desired location on the body of the user. For example, all of the body-associated detectors may be present on the same side of a body, such as the front torso of a body, or they may be present on opposite sides of a body, such as the front and back of the torso of a body. In the specific examples where the detector 20 is receiving acoustic information from within the lungs, which information is associated with the user inhaling, then the detectors are positions about the torso, proximal to the lungs. In accordance with another aspect of the present invention, where the detector 20 includes the accelerometer 90 that monitors the motion of the lungs associated with a rapid inhale, then the detectors are positioned close to the lungs. If the detector 20 has also detected the presence of a current indicative of the user holding the device 30, then the detector 20 records the occurrence of the event associated with the motion of the lungs. In such an example, the detector 20 would be positioned on the user's body in a location that allows best detection of the lung's motion.

Depending on the needs of a particular application, the current detected by the detector 20 may be generic, such that it merely identifies that the device 30 has contacted the target sites, which is the user's mouth and limb. In these instances, each device 30 can encode unique information in the current flow that uniquely identifies that particular device relative to all the other devices, especially if the user is using multiple devices.

The device 30 is designed to generate a variety of different types of signals, including but not limited to: current signatures produced through controlling conductance, RF signals, magnetic signals, conductive (near field) signals, acoustic signals, etc. The transmission time may vary, where in certain instances the transmission time may range from 0.1 sec to 48 hours or longer, including from 1 minute to 10 minutes. Depending on the given aspect, the identifier may produce a unique current signature once. Alternatively, the identifier may be configured to produce a unique current signature with the same information (identical signals), two or more times, where the collection of discrete identical signals may be collectively referred to as a redundant signal.

Depending on the particular application, the detector may be positioned in a variety of different configurations relative to the organ of interest. For example, where a single body-associated signal detector is employed, the methods may include initially positioning or implanting the single receiver at a location proximal to the organ of interest. Where the organ of interest is the lung, the single receiver may be positioned near the lungs, as desired. With other systems that include two or more signal detectors, the detectors may be positioned at a variety of body locations. For example, the methods may include positioning two or more distinct detectors at distinct locations near the lungs or positioning one detector at a front abdominal location and a second detector at a back location. This latter configuration is representative of instances where the detectors are placed on opposite sides of a target organ, e.g., to measure impedance through the organ, motion, or sound.

In various other aspects, the present invention provides a device to deliver an inhalable dose to a user and to confirm delivery of the dose. The device comprises a housing defining a chamber to store the dose, a power source secured to the housing and including a positive terminal and a negative terminal, a control module electrically coupled to the power source, an acoustic detector secured to the housing and electrically coupled to the control module, wherein the acoustic detector detects vibrations and at least two contact areas positioned on the housing such that the contact areas are at least partially exposed on the exterior of the housing and the at least two contact areas are electrically isolated from each other. At least one contact area is electrically coupled to one terminal of the power source and at least one other contact area is electrically coupled to the other terminal, and wherein a current path is completed through the user's body as the user makes contact with each of the two contact areas and current flow is detected by the control module.

In one aspect, the control module is electrically connected between the power source and one of the at least two contact areas, such that the control module is configured to control conductance to encode information in the current flow.

In one aspect, the control module is electrically coupled to the at least two contact areas and to both terminals of the power source.

In one aspect, the acoustic detector is secured to the outside of the housing.

In one aspect, a memory unit electrically connected to the control module to store information associated with delivery of the dose to the user.

In one aspect, a transceiver is electrically coupled to the control module such that information can be transmitted from the device to an external computer other than through the current flow and information can be received by the device.

In one aspect, the acoustic detector is secured inside the housing.

In one aspect, the acoustic detector provides an activation signal to the control module upon detection of a vibration representing the loading of the medication into the chamber.

In one aspect, the acoustic detector provides an activation signal to the control module and the control module records acoustic information associated with the user inhaling, wherein the information is provided to the control module through the acoustic detector.

In one aspect, the control module provides a unique time stamp associated with the delivery of the dose and wherein the control module receives an identifier signal that is associated with the user such that the combination of the time stamp and the identifier signal confirms deliver of the dose to the user.

In another aspect, a device is provided to deliver a dose to a user and track the timing of the delivery of the dose. The device comprises a housing defining a chamber to store the dose, at least two contact areas positioned on the housing such that the contact areas are at least partially exposed on the exterior of the housing and the at least two contact areas are electrically isolated from each other, a power source secured within the housing to provide power to produce a current flow through the user's body. The power source includes two terminals and each terminal is electrically coupled to one contact area. A control module is electrically coupled to the power source. The control module enters a sleep mode while the device is inactive. An acoustic detector is secured to the housing and is electrically coupled to the control module. The acoustic detector detects vibrations and sends an activation signal to the control module to activate the control module. The sound of the dose being loaded into the chamber activates the control module and wherein a current path is completed through the user's body as the user makes contact with each of the contact areas indicating that the user is about the receive a dose of the medication.

In one aspect, the device of further comprises a memory unit electrically coupled to the control module and secured to the housing, wherein the memory module stores information associated with delivery of the dose to the user.

In one aspect, the device further comprises a transceiver unit electrically coupled to the control module and secured to the housing. The transceiver unit allows the device to transmit and receive information.

In one aspect, the device further comprises an optical detector module for detecting that a dose was delivered to the user and the optical information is compared to acoustic information associated with the sound vibrations generated from inhaling through the device to confirm delivery of the dose to the user.

In yet another aspect, a system is provided to deliver an inhalable dose to a user and to confirm delivery of the dose. In one aspect, the system comprises a detector including a capacitive coupler, where the detector is worn by the user and a device comprising a housing defining a chamber to store the dose, a power source secured to the housing and including a positive terminal and a negative terminal, wherein the power source includes an isolating source that produces a carrier wave, a control module electrically coupled to the power source, wherein the control module alters the characteristics of the isolating source to encode information in the carrier wave; and at least two areas positioned on the housing such that one area is a partially exposed contact area and one area is capacitive coupled area, wherein the two areas are electrically isolated from each other. One output of the isolating source is coupled to the contact area and the other output of the isolating source is coupled to the capacitive coupled area. The contact area is touched by the user and the capacitive coupled area is capacitively coupled to the capacitive coupler worn by the user. A portion of the carrier wave's path is through the user's body using the contact area and a portion of carrier wave's path is through capacitive conductance using the capacitive coupling between the capacitive coupled area and the capacitive coupler worn by the user.

In one aspect, the housing defines an aperture to generate an acoustic wave as the user inhales through device and wherein the detector further comprises an acoustic detector for detecting acoustic wave associated with the user inhaling through the device, which acoustic waves traveling through the user's body and through the air, such that the acoustic detector correlates the acoustic wave through the user's body with the acoustic wave through the air to confirm delivery of the dose to the user.

It is to be understood that the various aspects of the present invention are not limited to particular aspects described, and as such may vary. It is also to be understood that the terminology used herein is for the purpose of describing particular aspects only, and is not intended to be limiting, since the scope of various aspects of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the various aspects of the present invention. The upper and lower limits of these smaller ranges may independently be included in the smaller ranges and are also encompassed within the various aspects of the present invention, subject to any specifically excluded limit in the stated range. Where the stated range includes one or both of the limits, ranges excluding either or both of those included limits are also included in the various aspects of the present the invention.

Unless defined otherwise, all technical and scientific terms used herein have the same meaning as commonly understood by one of ordinary skill in the art to which the various aspects of the present invention belongs. Although any methods and materials similar or equivalent to those described herein can also be used in the practice or testing of the various aspects of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the various aspects of the present invention are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Certain ranges have been presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As will be apparent to those of skill in the art upon reading this disclosure, each of the individual aspects described and illustrated herein has discrete components and features which may be readily separated from or combined with the features of any of the other several aspects without departing from the scope of the various aspects of the present invention. Any recited method can be carried out in the order of events recited or in any other order which is logically possible.

Although the foregoing aspects of the present invention have been described in some detail by way of illustration and example for purposes of clarity of understanding, it is readily apparent to those of ordinary skill in the art in light of the teachings of the various aspects of the present invention that certain changes and modifications may be made thereto without departing from the scope of the appended claims.

What is claimed is:

1. A method, comprising:
detecting, by a sensing and data acquisition circuit module, at least one parameter associated with an access port.

2. The method of claim 1, further comprising recording, by the sensing and data acquisition circuit module, the at least one parameter associated with the access port.

3. The method of claim 1, further comprising reporting, by the sensing and data acquisition circuit module, the at least one detected parameter associated with the access port.

4. The method of claim 1, further comprising providing an alert, by the sensing and data acquisition circuit module, based on the at least one detected parameter associated with the access port.

5. The method of claim 1, wherein the detecting of the at least one parameter associated with the access port comprises detecting a presence of a cleaning solution at the site of the access port.

6. The method of claim 5, wherein the detecting the presence of a cleaning solution comprises detecting via at least one of chemical detection, temperature detection, impedance detection, dielectric detection, and ultrasonic detection.

7. The method of claim 1, wherein the detecting of the at least one parameter associated with the access port comprises detecting an action associated with cleaning at least a portion of the access port.

8. The method of claim 7, wherein the detecting of the action associated with the cleaning of at least a portion of the access port comprises detecting via at least one of acoustic detection, accelerometer detection, optical detection, and magnetic detection.

9. An system for detecting at least one parameter associated with an access port, the system, comprising:
a sensing and data acquisition circuit module configured to detect at least one parameter associated with an access port, the sensing and data acquisition circuit module further comprising:
at least one sensor to sense the at least one parameter associated with the access port and generate a signal corresponding to the at least one parameter associated with the access port; and
a processor coupled to the sensor to process the signal generated by the sensor.

10. The system of claim 9, further comprising a memory coupled to the processor to record the at least one parameter associated with the access port.

11. The system of claim 9, further comprising a transceiver to report the at least one detected parameter associated with the access port.

12. The system of claim 9, further comprising an output circuit coupled to the processor to provide an alert based on the at least one detected parameter associated with the access port.

13. The system of claim 9, wherein the at least one sensor is configured to detect a presence of a cleaning solution at the site of the access port.

14. The system of claim 13, wherein the at least one sensor is selected from the group consisting of a chemical sensor, a temperature sensor, an impedance sensor, a dielectric sensor, and an ultrasonic sensor.

15. The system of claim 9, wherein the at least one sensor is configured to detect an action associated with cleaning at least a portion of the access port.

16. The system of claim 15, wherein the at least one sensor is selected from the group consisting of an acoustic sensor, an accelerometer sensor, an optical sensor, and a magnetic sensor.

17. A method, comprising:
wiping at least a portion of an access port;
allowing the access port to dry for a predetermined amount of time;
accessing the access port with a syringe;
detecting, by a sensing and data acquisition circuit module, at least one parameter associated with an access port.

18. The method of claim 17, further comprising recording, by the sensing and data acquisition circuit module, the at least one detected parameter associated with the access port.

19. The method of claim 17, further comprising reporting, by the sensing and data acquisition circuit module, the at least one detected parameter associated with the access port.

20. The method of claim 17, further comprising providing, by the sensing and data acquisition circuit module, an alert based on the at least one detected parameter.
